(19) United States
(12) Patent Application Publication
(10) Pub. No.: US 2018/0118856 A1
(43) Pub. Date: May 3, 2018

(54) RUBBER COMPOSITION AND TIRE

(71) Applicant: THE YOKOHAMA RUBBER CO., LTD., MINATO-KU, TOKYO (JP)

(72) Inventors: Manabu KATO, HIRATSUKA CITY, KANAGAWA (JP); Ryota TAKAHASHI, HIRATSUKA CITY, KANAGAWA (JP); Takahiro OKAMATSU, HIRATSUKA CITY, KANAGAWA (JP); Yoshiaki KIRINO, HIRATSUKA CITY, KANAGAWA (JP); Hidekazu ONOI, HIRATSUKA CITY, KANAGAWA (JP)

(21) Appl. No.: 15/565,567

(22) PCT Filed: Apr. 13, 2016
(86) PCT No.: PCT/JP2016/061881

§ 371 Date: Oct. 10, 2017

(30) Foreign Application Priority Data
Apr. 13, 2015 (JP) 2015-081857

(51) Int. Cl. C08C 19/22; C08L 13/00; C08L 15/00; C08L 83/04; C08K 3/36; C07C 291/02; B60C 1/00

(52) U.S. Cl. CPC C08C 19/22; C08L 13/00; C08L 15/00; B60C 1/00; C08K 3/36; C07C 291/02; C08L 83/04

(57) ABSTRACT

The present invention provides a rubber composition and a tire, which provides superior wear resistance and processability while maintaining the wet performance. The present invention provides a rubber composition for a tire including a rubber component including a modified diene rubber with carboxy group modification, silica and a predetermined polysiloxane; wherein the content of the silica is from 60 to 200 parts by mass per 100 parts by mass of the rubber component, the content of the polysiloxane is from 1 to 20 mass % based on the content of the silica, and the content of the modified diene rubber is from 10 to 100 parts by mass per 100 parts by mass of the rubber component. The present invention also provides a tire using the rubber composition.

### TECHNICAL FIELD

The present invention relates to a rubber composition and a pneumatic tire.

### BACKGROUND ART

Conventionally, superior wet performance, low heat build-up, wet performance and the like are required for a tire. However, improvement of wet performance and low heat build-up may be achieved while sacrificing wear resistance.

Therefore, Patent Document 1 is proposed, aiming to provide a rubber composition having excellent wet performance and wear resistance when formed into a tire as well as excellent processability.

Patent Document 1 proposes a rubber composition for a tire including: a silica at from 60 to 200 parts by mass per 100 parts by mass of a diene rubber; polysiloxane represented by Formula (1) below as a sulfur-containing silane coupling agent at from 1 to 20 mass % of the content of the silica; and thiuram disulfide-type vulcanization accelerator represented by Formula (I) below at from 0.05 to 3.0 parts by mass;

(A)ₐ(B)_{b}(C)_{c}(D)_{d}(R¹)ₑSiO_{(4-2a-b-c-d-e)/2} (1)

Formula (1) is an average composition formula, wherein A is a divalent organic group containing a sulfide group; B is a monovalent hydrocarbon group having from 5 to 10 carbons; C is a hydrolyzable group; D is an organic group containing a mercapto group; R¹ is a monovalent hydrocarbon group having from 1 to 4 carbons; and a to e satisfy the relational expressions 0≤a<1, 0≤b<1, 0<c<3, 0<d<1, 0≤e<2, and 0<2a+b+c+d+e<4, and at least one of a and b is not 0.

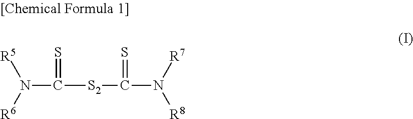

wherein each R⁵, R⁶, R⁷ and R⁸ is independently a hydrocarbon group having from 2 to 18 carbons.

### CITATION LIST

### Patent Literature

Patent Document 1: WO 2014/129662

### SUMMARY OF INVENTION

### Technical Problem

The present inventors evaluated the rubber composition according to the Patent Document 1 and found that such a rubber composition tends to show decrease in processability and has a room for further improvement in wear resistance.

Therefore, in light of the circumstances described above, an object of the present invention is to provide a rubber composition having excellent wear resistance while maintaining excellent wet performance when formed into a tire as well as excellent processability.

Another object of the present invention is to provide a tire.

### Solution to Problem

The present inventors conducted a diligent research to solve the above problems, and discovered that a rubber composition including a modified diene rubber modified by a carboxy group at a predetermined degree of modification can exhibit a predetermined effect, thus completed the present invention.

The present invention is based on the knowledge described above and specifically solves the problem above by the configuration below.

1. A rubber composition for a tire including:

a rubber component including a modified diene rubber, the modified diene rubber having from 0.2 to 4 mol % of all the double bonds in a raw material diene rubber modified by a carboxy group;

a silica; and

polysiloxane represented by an average compositional formula (I) below;

wherein a content of the silica is from 60 to 200 parts by mass per 100 parts by mass of the rubber component, a content of the polysiloxane is from 1 to 20 mass % based on the content of the silica, and a content of the modified diene rubber is from 10 to 100 parts by mass per 100 parts by mass of the rubber component;

(A)ₐ(B)_{b}(C)_{c}(D)_{d}(R¹)ₑSiO_{(4-2a-b-c-d-e)/2} (I)

where, in the average compositional formula (I), A is a divalent organic group containing a sulfide group; B is a monovalent hydrocarbon group having from 5 to 10 carbons; C is a hydrolyzable group; D is an organic group containing a mercapto group; R¹ is a monovalent hydrocarbon group having from 1 to 4 carbons; and a to e satisfy the relational expressions 0≤a<1, 0≤b<1, 0<c<3, 0<d<1, 0≤e<2, and 0<2a+b+c+d+e<4, and at least one of a and b is not 0.

2. The rubber composition according to 1 above, wherein the modified diene rubber is produced by a reaction between the raw material diene rubber and a nitrone compound having a carboxy group and a nitrone group.

3. The rubber composition according to 2 above, wherein

the nitrone compound is at least one type selected from the group consisting of N-phenyl-α-(4-carboxyphenyl)nitrone, N-phenyl-α-(3-carboxyphenyl)nitrone, N-phenyl-α-(2-carboxyphenyl)nitrone, N-(4-carboxyphenyl)-α-phenylnitrone, N-(3-carboxyphenyl)-α-phenylnitrone, and N-(2-carboxyphenyl)-α-phenylnitrone.

4. The rubber composition according to 2 or 3 above, wherein a content of the nitrone compound introduced in the modified diene rubber is not less than 0.3 parts by mass and not greater than 10 parts by mass per 100 parts by mass of the rubber component.

5. A rubber composition for a tire including:

a rubber component including a modified diene rubber, the modified diene rubber having a double bond and a carboxy group, and a content of the carboxy group being from 0.2 to 4 mol % of a total of the double bond and the carboxy group;

a silica; and

polysiloxane represented by an average compositional formula (I) below;

wherein a content of the silica is from 60 to 200 parts by mass per 100 parts by mass of the rubber component, a content of the polysiloxane is from 1 to 20 mass % based on the content of the silica, and a content of the modified diene rubber is from 10 to 100 parts by mass per 100 parts by mass of the rubber component;

(A)ₐ(B)_{b}(C)_{c}(D)_{d}(R¹)ₑSiO_{(4-2a-b-c-d-e)/2} (I)

where, in the average compositional formula (I), A is a divalent organic group containing a sulfide group; B is a monovalent hydrocarbon group having from 5 to 10 carbons; C is a hydrolyzable group; D is an organic group containing a mercapto group; R¹ is a monovalent hydrocarbon group having from 1 to 4 carbons; and a to e satisfy the relational expressions 0≤a<1, 0≤b<1, 0<c<3, 0<d<1, 0≤e<2, and 0<2a+b+c+d+e<4, and at least one of a and b is not 0.

6. A tire including the rubber composition described in any one of 1 to 5.

Note that the modified diene rubber included in the rubber composition described in 5 above corresponds to the modified diene rubber included in the rubber composition described in 1 above. In the present invention, the modified diene rubber may be one of the modified diene rubber included in the rubber composition described in 5 above and the modified diene rubber included in the rubber composition described in 1 above.

Also, each component in the rubber composition described in 5 above other than the modified diene rubber is the same as the component in the rubber composition described in 1 above other than the modified diene rubber.

### Advantageous Effects of Invention

The rubber composition of the present invention exhibits excellent wear resistance while maintaining excellent wet performance, and has excellent processability.

The tire of the present invention exhibits excellent wear resistance while maintaining excellent wet performance, and has excellent processability.

### BRIEF DESCRIPTION OF DRAWING

FIG. 1 is a partial cross-sectional schematic view of a tire that represents one embodiment of the tire of the present invention.

### DESCRIPTION OF EMBODIMENTS

Embodiments of the present invention are described in detail below.

Note that, in the present specification, numerical ranges indicated using “(from) . . . to . . . ” include the former number as the lower limit value and the later number as the upper limit value.

Also, when a component includes two or more types of substances, the content of the component is a total content of two or more types of the substances.

The rubber composition of the present invention is a rubber composition for a tire including:

a rubber component including a modified diene rubber, the modified diene rubber having from 0.2 to 4 mol % of all the double bonds in a raw material diene rubber modified by a carboxy group;

a silica; and

polysiloxane represented by an average compositional formula (I) below;

wherein a content of the silica is from 60 to 200 parts by mass per 100 parts by mass of the rubber component, a content of the polysiloxane is from 1 to 20 mass % based on the content of the silica, and a content of the modified diene rubber is from 10 to 100 parts by mass per 100 parts by mass of the rubber component.

(A)ₐ(B)_{b}(C)_{c}(D)_{d}(R¹)ₑSiO_{(4-2a-b-c-d-e)/2} (I)

wherein, in the average compositional formula (I), A is a divalent organic group containing a sulfide group; B is a monovalent hydrocarbon group having from 5 to 10 carbons; C is a hydrolyzable group; D is an organic group containing a mercapto group; R¹ is a monovalent hydrocarbon group having from 1 to 4 carbons; and a to e satisfy the relational expressions 0≤a<1, 0≤b<1, 0<c<3, 0<d<1, 0≤e<2, and 0<2a+b+c+d+e<4, and at least one of a and b is not 0.

The rubber composition of the present invention is thought to achieve desired effects as a result of having such a configuration. Although the reason for this is unknown, the reason is presumed to be as follows.

Because the modified diene rubber, which is modified by a carboxy group at a predetermined degree of modification, can interact and/or form a bond with a silica, such a modified diene rubber can promote the dispersion of the silica when the modified diene rubber and a predetermined polysiloxane (as a silane coupling agent) are used together.

It is surmised that the rubber composition can exhibit improved wear resistance while maintaining the excellent wet performance because the silica and the modified diene rubber can interact and/or form a bond each other as described above.

Also, the content of silica can be increased in the present invention because of the excellent dispersibility of silica. It is surmised that the rubber composition exhibits excellent processability because the interaction via the carboxy group is reversible even if the content of silica is increased.

### Rubber Composition

Each of the components included in the rubber composition of the present invention will be described in detail below.

### Rubber Component

The rubber component included in the rubber composition of the present invention contains the modified diene rubber.

### Modified Diene Rubber

The rubber component includes the modified diene rubber, in which from 0.2 to 4 mol % of the total double bonds in the raw material diene rubber is modified to a carboxy group.

Note that the ratio of the carboxy groups included in the modified diene rubber in moles to the total double bonds included in the raw material diene rubber in moles, or the ratio of the carboxy groups in moles to the total of the double bonds and the carboxy groups included in the modified diene rubber in moles is sometimes referred to as a degree of modification in the present specification. That is, the degree of modification in the present invention is from 0.2 to 4 mol %.

Also, in the present invention, the modified diene rubber has a double bond and a carboxy group, and the content of the carboxy group is from 0.2 to 4 mol % of the total of the double bonds and carboxy groups.

In the present invention, the modified diene rubber has a carboxy group as a modified group.

### Modified Group

The modified diene rubber may have a carboxy group as a modified group in at least one selected from the group consisting of the main chain and the side chain. The modified diene rubber may have a carboxy group as a modified group in at least one selected from the group consisting of a part of the main chain and a part of the side chain.

Examples of the modified group in the main chain are, for example, those expressed by Formula (II) below.

Examples of the modified group in the side chain are, for example, those expressed by Formula (III) below.

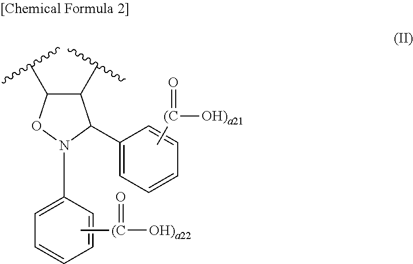

In Formula (II) above, each a21 and a22 is independently preferably from 0 to 5, and more preferably 0, 1 or 2.

For the value of a21+a22, 1 or more is preferable, from 1 to 4 is more preferable and from 1 to 2 is even more preferable.

Each a21, a22 and a21+a22 in Formula (II) is the same as n, m, m+n, respectively, in Formula (3) described below.

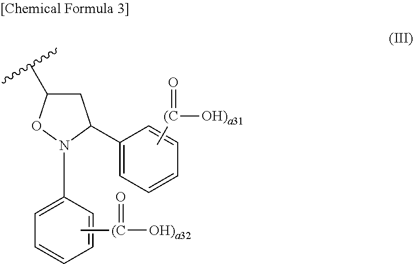

In Formula (III) above, each a31 and a32 is independently preferably from 0 to 5, and more preferably 0, 1 or 2.

For the value of a31+a32, 1 or more is preferable, from 1 to 4 is more preferable and from 1 to 2 is even more preferable.

Each a31, a32 and a31+a32 in Formula (III) is the same as n, m, m+n, respectively, in Formula (3) described below.

Examples of the main chain of the modified diene rubber include the same main chain in the diene rubber used as a raw material diene rubber described below. Among these, an aromatic vinyl-conjugated diene copolymer rubber is preferable and a styrene-butadiene rubber is more preferable from the perspective of excellent strength characteristics and low heat build-up.

### Method of Producing Modified Diene Rubber

The modified diene rubber produced by the reaction between the raw material diene rubber and a modifying agent having a carboxy group is preferable because the modified diene rubber is superior in at least one effect selected from the group consisting of wet performance, wear resistance and processability (referred to as “exhibiting superior effect of the present invention” hereinafter).

The modified diene rubber is preferably modified to a carboxy group in at least one or both of the main chain and the side chain.

### Raw Material Diene Rubber

The diene rubber used as the raw material diene rubber is not particularly limited. Examples include natural rubber (NR), isoprene rubber (IR), aromatic vinyl-conjugated diene copolymer rubber, acrylonitrile-butadiene copolymer rubber (NBR), butyl rubber (IIR), butyl halide rubber (Br—IIR, Cl—IIR), and chloroprene rubber (CR). Among these, an aromatic vinyl-conjugated diene copolymer rubber is preferable and a styrene-butadiene rubber is more preferable from the perspective of excellent strength characteristics and low heat build-up.

The styrene-butadiene rubber that can be used as a raw material diene rubber may not be particularly limited as long as it is a copolymer of styrene and butadiene. The styrene-butadiene rubber exhibits an excellent reactivity toward the modifying agent due to small steric hindrance of the unsaturated bond derived from the butadiene.

The styrene content in the styrene-butadiene rubber is preferably not less than 10 mass % and more preferably from 26 to 70 mass %, from the perspective of excellent compatibility with an modifying agent.

The “styrene content in the styrene-butadiene rubber” herein refers to the proportion (mass % or wt. %) of the styrene units in the total units configuring styrene-butadiene rubber.

In the present invention, the microstructure of the styrene-butadiene rubber is measured in accordance with JIS K 6239:2007 (Rubber, raw, S-SBR Determination of the microstructure).

The double bond derived from butadiene, which is present in the styrene-butadiene rubber, includes a 1,4 bond (cis-1,4 bond, trans-1,4 bond) and 1,2 bond.

The proportion of the 1,4 bonds in the double bonds present in the styrene-butadiene rubber is preferably from 20 to 80 mol % and more preferably from 25 to 65 mol %, to the total double bonds.

The “proportion of the 1,4 bonds in the double bonds present in the styrene-butadiene rubber” herein refers to a proportion (in mol %) of the 1,4 bonds in the total double bonds present in the styrene-butadiene rubber (trans-1,4 unit, cis-1,4 unit and 1,2 unit, in the butadiene component and so forth).

The proportion of the 1,2 bonds in the double bonds present in the styrene-butadiene rubber (vinyl content or vinyl bond content) is preferably from 20 to 80 mol % and more preferably from 35 to 75 mol %, to the total double bonds.

The “proportion of the 1,2 bonds in the double bonds present in the styrene-butadiene rubber” herein refers to a proportion (in mol %) of the 1,2 units (1,2 bonds) in the total double bonds present in the styrene-butadiene rubber.

From the viewpoint of handling, the raw material diene rubber preferably has a weight average molecular weight (Mw) of from 100000 to 1500000, and more preferably from 100000 to 1400000 and even more preferably from 300000 to 1300000. The weight average molecular weight (Mw) of the raw material diene rubber is measured by gel permeation chromatography (GPC) on the basis of polystyrene standard using tetrahydrofuran as a solvent.

### Modifying Agent

The modifying agent that can be used during the production of the modified diene rubber is described below. The modifying agent is preferably a compound including a carboxy group and more preferably a nitrone compound including a carboxy group and a nitrone group.

The number of the carboxy groups present in the modifying agent per molecule is preferably not less than 1 and can be not greater than 10, and more preferably from 1 to 4, and even more preferably from 1 to 2.

The nitrone group is a group represented by Formula (1) below.

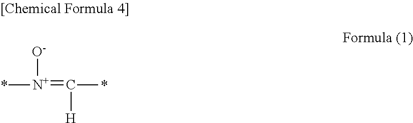 [SMILES: */C=[N+](/*)[O-]]

In Formula (1), * represents a bonding position.

The number of the nitrone groups in the modifying agent per molecule is preferably from 1 to 3.

The modifying agent is preferably a compound represented by Formula (2) below.

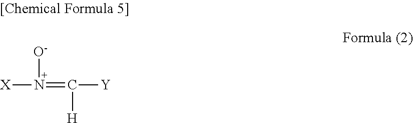

In Formula (2) above, X and Y each independently represent an aliphatic hydrocarbon group, an aromatic hydrocarbon group, or an aromatic heterocycle group that may have a substituent. The carboxy group can bond to either one of or both of X and Y.

Examples of the aliphatic hydrocarbon group represented by X or Y include alkyl groups, cycloalkyl groups, alkenyl groups, and the like.

Examples of the alkyl group include methyl groups, ethyl groups, n-propyl groups, isopropyl groups, n-butyl groups, isobutyl groups, sec-butyl groups, tert-butyl groups, n-pentyl groups, isopentyl groups, neopentyl groups, tert-pentyl groups, 1-methylbutyl groups, 2-methylbutyl groups, 1,2-dimethylpropyl groups, n-hexyl groups, n-heptyl groups, and n-octyl groups.

Among these, alkyl groups having from 1 to 18 carbons are preferable, and alkyl groups having from 1 to 6 carbons are more preferable.

Examples of the cycloalkyl group include cyclopropyl groups, cyclobutyl groups, cyclopentyl groups, and cyclohexyl groups.

Among these, cycloalkyl groups having from 3 to 10 carbons are preferable, and cycloalkyl groups having from 3 to 6 carbons are more preferable.

Examples of the alkynyl group include vinyl groups, 1-propenyl groups, allyl groups, isopropenyl groups, 1-butenyl group, and 2-butenyl groups.

Among these, alkenyl groups having from 2 to 18 carbons are preferable, and alkenyl groups having from 2 to 6 carbons are more preferable.

Examples of the aromatic hydrocarbon group represented by X or Y include aryl groups, aralkyl groups, and the like.

Examples of the aryl group include a phenyl group, a naphthyl group, an anthryl group, a phenanthryl group, a biphenyl group, and the like. Among these, aryl groups having from 6 to 14 carbons are preferable, aryl groups having from 6 to 10 carbons are more preferable, and a phenyl group and a naphthyl group are even more preferable.

Examples of the aralkyl group include benzyl groups, phenethyl groups, and phenylpropyl groups. Among these, aralkyl groups having from 7 to 13 carbons are preferable, aralkyl groups having from 7 to 11 carbons are more preferable, and benzyl groups are even more preferable.

Examples of the aromatic heterocyclic group represented by X or Y include pyrrolyl groups, furyl groups, thienyl groups, pyrazolyl groups, imadazolyl groups (imadazol groups), oxazolyl groups, isooxazolyl groups, thiazolyl groups, isothiazolyl groups, pyridyl groups (pyridine groups), furan groups, thiophene groups, pyridazinyl groups, pyrimidinyl groups, and pyradinyl groups. Among these, pyridyl groups are preferable.

The substituents of the group other than the carboxy group represented by X or Y are not particularly limited, and examples thereof include alkyl groups having from 1 to 4 carbons, hydroxy groups, amino groups, nitro groups, sulfonyl groups, alkoxy groups, and halogen atoms.

Note that examples of the aromatic hydrocarbon group having such a substituent include aryl groups having an alkyl group, such as a tolyl group and xylyl group; and aralkyl groups having a substituent, such as a methylbenzyl group, ethylbenzyl group, and methylphenethyl group.

The modifying agent is preferably a compound represented by Formula (3) below, from the viewpoint of excellent compatibility and reactivity with the raw material diene rubber.

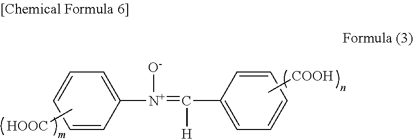

In Formula (3), m and n each independently represent an integer of 0 to 5, and the sum of m and n is 1 or greater.

The integer represented by m is preferably an integer of 0 to 2, and more preferably an integer of 0 or 1, because solubility to a solvent during modifying agent synthesis will be better and thus synthesis will be easier.

The integer represented by n is preferably an integer of 0 to 2, and more preferably an integer of 0 or 1, because solubility to a solvent during modifying agent synthesis will be better and thus synthesis will be easier.

Furthermore, the sum of m and n (m+n) is preferably from 1 to 4, and more preferably 1 or 2.

The modifying agent is not particularly limited but is preferably one type of compound selected from the group consisting of N-phenyl-α-(4-carboxyphenyl)nitrone represented by Formula (3-1) below, N-phenyl-α-(3-carboxyphenyl)nitrone represented by Formula (3-2) below, N-phenyl-α-(-2-carboxyphenyl)nitrone represented by Formula (3-3) below, N-(-4-carboxyphenyl)-α-phenylnitrone represented by Formula (3-4) below, N-(-3-carboxyphenyl)-α-phenylnitrone represented by Formula (3-5) below, and N-(2-carboxyphenyl)-α-phenylnitrone represented by Formula (3-6) below.

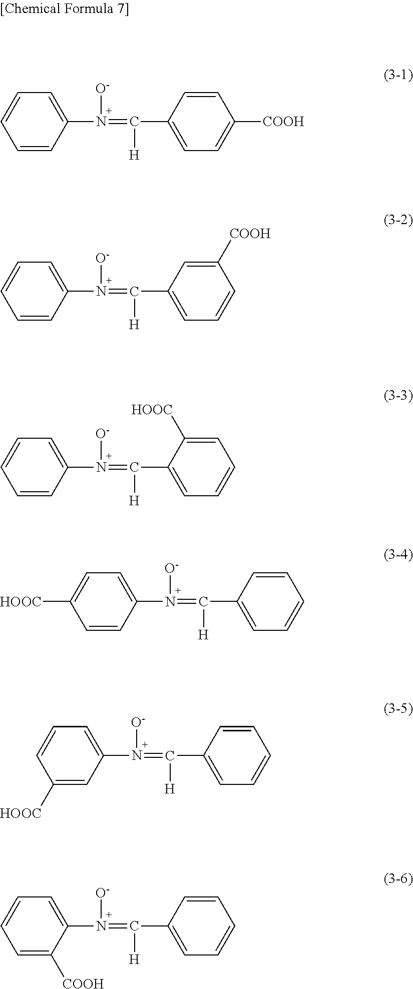

The method of synthesizing the modifying agent is not particularly limited, and conventionally known methods can be used. For example, a compound having a nitrone group can be produced by stirring a compound having a hydroxyamino group (—NHOH) and a compound having an aldehyde group (—CHO) at a molar ratio of hydroxyamino group to aldehyde group (—NHOH/—CHO) of from 1.0 to 1.5 in the presence of an organic solvent (for example methanol, ethanol, and tetrahydrofuran) at room temperature for from 1 to 24 hours to allow the both groups to react. Either one or both of the compound having a hydroxyamino group and the compound having an aldehyde group described above may have a carboxy group. If the modifying agent has a substituent besides the carboxy group, either one or both of the compound having a hydroxyamino group and the compound having an aldehyde group can have the substituent above.

The method of producing a modified diene rubber is not limited to a particular method. Examples of the method include blending the raw material diene rubber and the modifying agent at a temperature of from 100 to 200° C. for from 1 to 30 minutes.

The amount of the modifying agent used during the production of the modified diene rubber is preferably from 0.1 to 10 parts by mass and more preferably from 0.3 to 5 parts by mass per 100 parts by mass of the raw material diene rubber.

Also, in the present invention, the modified diene rubber is a modified diene rubber, in which from 0.2 to 4 mol % of the total double bonds present in the raw material diene rubber is modified to carboxy groups. Alternatively, the modified diene rubber is a modified diene rubber, in which the double bonds and carboxy groups are present, and the content of the carboxy group is from 0.2 to 4 mol % of the total of the double bonds and carboxy groups.

That is, the degree of modification in the present invention is from 0.2 to 4 mol %. The degree of modification above is preferably from 0.2 to 1.0 mol % and more preferably from 0.3 to 0.8 mol % from the viewpoint of exhibiting superior effect of the present invention and increase in the vulcanization rate. The degree of modification above is preferably from 0.4 to 0.8 mol % from the viewpoint of increase in the vulcanization rate.

In the present invention, the degree of modification can be determined by the NMR (Nuclear Magnetic Resonance) of the raw material diene rubber and the modified diene rubber, for example.

Specifically, the raw material diene rubber and modified diene rubber are measured for the peak area at around 8.08 ppm (assigned to two protons adjacent to the carboxy group) by ¹H-NMR (CDCl₃, 400 MHz, TMS (tetramethylsilane)) using CDCl₃ as a solvent. Specifically, when the carboxy group is bonded to the benzene ring, the peak area assigned to two protons bonded to the carbon atom adjacent to the carbon atom bonded to the carboxy group is measured to determine the degree of modification.

The content of the modifying agent (e.g. nitrone compound) introduced in the modified diene rubber is preferably from 0.3 to 10 parts by mass and more preferably from 0.3 to 5 parts by mass per 100 parts by mass of the rubber component, from the viewpoint of exhibiting superior effect of the present invention.

The content of the modifying agent introduced in the modified diene rubber is preferably from 0.5 to 10 parts by mass and more preferably from 0.5 to 5 parts by mass per 100 parts by mass of the rubber component, from the viewpoint of excellent processability and an increase in the vulcanization rate.

A single modified diene rubber can be used, or a combination of two or more types can be used.

In the present invention, the content of the modified diene rubber is from 10 to 100 parts by mass per 100 parts by mass of the rubber component. The content of the modified diene rubber is preferably from 20 to 90 parts by mass, and more preferably from 50 to 80 parts by mass per 100 parts by mass of the rubber component.

In the present invention, the rubber component can include a rubber besides the modified diene rubber. Examples of the rubber besides the modified diene rubber include a diene rubber. There is no particular limitation on such a diene rubber. Examples of the diene rubber include the same raw material diene rubber used during the production of the modified diene rubber.

The diene rubber is preferably at least one selected from natural rubber, styrene butadiene rubber, and butadiene rubber.

There is no limitation on natural rubber, styrene butadiene rubber, and butadiene rubber. For example, they may be used in the same manner as the raw material diene rubber.

### Silica

The silica included in the rubber composition of the present invention is not particularly limited, and any conventionally known silica that is compounded into a rubber composition in applications such as tires can be used.

Specific examples of the silica include fumed silica, calcined silica, precipitated silica, pulverized silica, molten silica, colloidal silica, and the like.

The CTAB adsorption specific surface area of the silica is preferably not less than 150 m²/g and more preferably from 155 to 230 m²/g, from the viewpoint of exhibiting superior effect of the present invention. The CTAB of the silica was measured in accordance with the CTAB adsorption method disclosed in JIS K6217-3:2001.

A single silica can be used, or a combination of two or more types can be used.

In the present invention, the content of the silica is from 60 to 200 parts by mass per 100 parts by mass of the rubber component. The content of the silica is preferably from 60 to 150 parts by mass per 100 parts by mass of the rubber component from the viewpoint of exhibiting superior effect of the present invention.

### Polysiloxane

The polysiloxane included in the rubber composition of the present invention is a compound represented by the average compositional formula (I) below.

(A)ₐ(B)_{b}(C)_{c}(D)_{d}(R¹)ₑSiO_{(4-2a-b-c-d-e)/2} (I)

wherein, in the average compositional formula (I), A is a divalent organic group containing a sulfide group; B is a monovalent hydrocarbon group having from 5 to 10 carbons; C is a hydrolyzable group; D is an organic group containing a mercapto group; R¹ is a monovalent hydrocarbon group having from 1 to 4 carbons; and a to e satisfy the relational expressions 0≤a<1, 0≤b<1, 0<c<3, 0<d<1, 0≤e<2, and 0<2a+b+c+d+e<4, and at least one of a and b is not 0 (excluding the case where both a and b are 0).

In the present invention, because the polysiloxane contains C, it has excellent affinity and/or reactivity with silica.

Because the polysiloxane contains D, it can interact and/or react with the diene rubber, which yields excellent wet performance and wear resistance.

When the polysiloxane has A, the wet performance, wear resistance and processability (in particular, the maintenance and prolongation of the Mooney scorch time) are superior.

When the polysiloxane has B, the mercapto group is protected, and the Mooney scorch time becomes longer, while at the same time, the processability is excellent due to outstanding affinity with the rubber.

The polysiloxane included in the rubber composition of the present invention has a siloxane skeleton as its skeleton. The siloxane skeleton can be either one of straight-chain, branched, or three-dimensional, or combination thereof.

In the average compositional formula (I) above, A is a divalent organic group containing a sulfide group (also called a sulfide group-containing organic group, hereafter). The organic group may be, for example, a hydrocarbon group optionally having a hetero atom such as an oxygen atom, a nitrogen atom, or a sulfur atom.

Among these, a group represented by Formula (4) below is preferable.

*—(CH₂)ₙ—Sₓ—(CH₂)ₙ—* (4)

In Formula (4) above, n represents an integer of 1 to 10, among which an integer of 2 to 4 is preferable.

In Formula (4) above, x represents an integer of 1 to 6, among which an integer of 2 to 4 is preferable.

In Formula (4) above, * represents a bonding position.

Specific examples of the group represented by Formula (4) above include *—CH₂—S₂—CH₂—*, *—C₂H₄—S₂—C₂H₄—*, *—C₃H₆—S₂—C₃H₆—*, *—C₄H₈—S₂—C₄H₈—*, *—CH₂—S₄—CH₂—*, *—C₂H₄—S₄—C₂H₄—*, *—C₃H₆—S₄—C₃H₆—*, * C₄H₈—S₄—C₄H₁₈—*, and the like.

In the average compositional formula (I), B is a monovalent hydrocarbon group having from 5 to 10 carbons, specific examples of which include hexyl groups, octyl groups, and decyl groups. Among these, B is preferably a monovalent hydrocarbon group having from 8 to 10 carbons from the perspective of protecting the mercapto group, having a long Mooney scorch time, having superior processability, and having superior wet performance, wear resistance and low rolling resistance.

In the average compositional formula (I) above, C represents a hydrolyzable group, and specific examples thereof include alkoxy groups, phenoxy groups, carboxyl groups, alkenyloxy groups, and the like. Among these, a group represented by Formula (5) below is preferable.

*—OR² (5)

In Formula (5) above, R² represents an alkyl group having from 1 to 20 carbons, an aryl group having from 6 to 10 carbons, an aralkyl group (aryl alkyl group) having from 7 to 10 carbons, or an alkenyl group having from 2 to 10 carbons. Among these, an alkyl group having from 1 to 5 carbons is preferable.

Specific examples of the alkyl group having from 1 to 20 carbons include a methyl group, ethyl group, propyl group, butyl group, hexyl group, octyl group, decyl group, octadecyl group, and the like.

Specific examples of the aryl group having from 6 to 10 carbons include a phenyl group, tolyl group, and the like.

Specific examples of the aralkyl group having from 7 to 10 carbons include a benzyl group, phenylethyl group, and the like.

Specific examples of the alkenyl group having from 2 to 10 carbons include vinyl groups, propenyl groups, and pentenyl groups.

In Formula (5) above, * represents a bonding position.

In the average compositional formula (I) above, D represents an organic group having a mercapto group. Among these, a group represented by Formula (6) below is preferable.

*—(CH₂)ₘ—SH (6)

In Formula (6), m is an integer of 1 to 10. Among these, m is preferably an integer of 1 to 5.

In Formula (6) above, * represents a bonding position.

Specific examples of the group represented by Formula (6) above include *—CH₂SH, *—C₂H₄SH, *—C₃H₆SH, *—C₄H₈SH, *—C₅H₁₀SH, *—C₆H₁₂SH, *—C₇H₁₄SH, *—C₈H₁₆SH, *—C₉H₁₈SH, and *—C₁₀H₂₀SH.

In the average compositional formula (I), R¹ is a monovalent hydrocarbon group having from 1 to 4 carbons. Examples of the hydrocarbon group R¹ include a methyl group, an ethyl group, a propyl group, and a butyl group.

In the average compositional formula (I), the values of a to e satisfy the relational expressions 0≤a<1, 0≤b<1, 0<c<3, 0<d<1, 0≤e<2, and 0<2a+b+c+d+e<4, and at least one of a and b is not 0. In one of the preferred aspects, both a and b are greater than 0.

The value of a of the polysiloxane is preferably greater than 0 (0<a) because a Mooney scorch time is longer and processability is superior. That is, a case in which the polysiloxane has a sulfide-containing organic group is a preferred aspect. Among these, from the perspective of achieving even better processability, superior wet performance and low rolling resistance, the value of a preferably satisfies 0<a≤0.50.

In addition, the value of a of the polysiloxane is preferably 0 from the perspective of superior wet performance, wear resistance, and low rolling resistance. That is, a case in which the polysiloxane does not have a sulfide-containing organic group is a preferred aspect.

In the average compositional formula (I), the value of b is preferably greater than 0 and preferably satisfies the expression 0.10≤b≤0.89 from the perspective of superior wet performance, processability, and low rolling resistance.

In the average compositional formula (I), the value of c preferably satisfies the expression 1.2≤c≤2.0 from the perspective of superior wet performance, processability, silica dispersibility, and low rolling resistance.

In the average compositional formula (I), the value of d preferably satisfies the expression 0.1≤d≤0.8 from the perspective of superior wet performance, processability, and low rolling resistance.

In the average compositional formula (I), the values of a to e preferably satisfy the relationship 0<2a+b+c+d+e≤3 from the perspective of superior wet performance, processability, and low rolling resistance.

From the perspective of achieving better dispersibility of silica and superior processability, the polysiloxane is preferably a polysiloxane having a group represented by Formula (4) above as A, a group represented by Formula (5) above as C, and a group represented by Formula (6) above as D, in the average compositional formula (I). In addition to the above reasons, from the perspective of protecting the mercapto group, having a long Mooney scorch time, having superior processability, and having superior wet performance and low rolling resistance, the polysiloxane more preferably has a group represented by Formula (4) above as A, a group represented by Formula (5) above as C, and a group represented by Formula (6) above as D, and a monovalent hydrocarbon group having from 8 to 10 carbons as B.

The weight average molecular weight of the polysiloxane is preferably from 500 to 2300 and more preferably from 600 to 1500, from the perspective of achieving superior wet performance, processability, and low rolling resistance. The molecular weight of the polysiloxane is the weight average molecular weight determined in terms of polystyrene by gel permeation chromatography (GPC) using toluene as a solvent.

The mercapto equivalent weight of the polysiloxane determined by the acetic acid/potassium iodide/potassium iodate addition-sodium thiosulfate solution titration method is preferably from 550 to 1900 g/mol, and more preferably from 600 to 1800 g/mol, from the perspective of having excellent vulcanization reactivity.

The method of producing the polysiloxane is not particularly limited. Examples thereof include conventionally known methods.

A single polysiloxane can be used or a combination of two or more polysiloxanes can be used.

In the present invention, the content of the polysiloxane is from 1 to 20 mass % based on the content of silica. The content of the polysiloxane is preferably from 3 to 18 mass % and more preferably from 4 to 18 mass % based on the content of silica from the viewpoint of exhibiting superior effect of the present invention.

### Terpene Resin

The rubber composition of the present invention may further contain terpene resin. Terpene resin may be a polymer at least using a terpene-based monomer as a monomer and it may be either a homopolymer or a copolymer. Also, the terpene resin may be modified by an aromatic compound, for example.

Examples of the terpene-based monomer include α-pinene, β-pinene, dipentene, limonene, and derivatives thereof.

Examples of the aromatic compound include styrene, α-methylstyrene, vinyl toluene, indene, and phenols.

The terpene resin may include an aromatic modified terpene resin. The terpene resin is preferably the aromatic modified terpene resin from the perspective of an increase in tan δ at 0° C. of the rubber composition and superior wet performance, wear resistance and excellent balance with the low rolling resistance, due to excellent compatibility with the diene rubber.

The softening point of the terpene resin (in particular, an aromatic modified terpene resin) is preferably from 60 to 150° C. and more preferably from 70 to 130° C. from the perspective of having superior wet performance, and wear resistance.

A method of producing the terpene resin is not particularly limited. Examples thereof include conventionally known methods. The terpene resin may be used alone or as a combination of two or more types.

The amount of the terpene resin is preferably from 1 to 30 parts by mass and more preferably from 3 to 20 parts by mass per 100 parts by mass of the rubber component.

### Thiuram Disulfide-Based Vulcanization Accelerator

The rubber composition of the present invention may further include a thiuram disulfide-based vulcanization accelerator from the perspective of exhibiting superior effect of the present invention. The thiuram disulfide-based vulcanization accelerator is not particularly limited. Specific examples include a compound represented by Formula (IV) below.

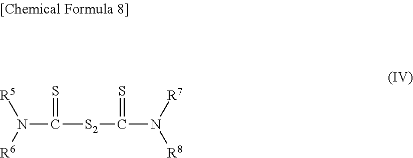

wherein each R⁵, R⁶, R⁷ and R⁸ is independently a hydrocarbon group having 2 to 18 carbon atoms. Examples of the hydrocarbon group include aliphatic hydrocarbon groups, alicyclic hydrocarbon groups, aromatic hydrocarbon groups, and combinations thereof. The hydrocarbon group may include a hetero atom such as an oxygen atom, nitrogen atom and sulfur atom, and may have an unsaturated bond. Examples of the hydrocarbon group include aliphatic hydrocarbon groups such as methyl groups, ethyl groups and butyl groups; cycloaliphatic hydrocarbon groups such as cyclohexyl groups, aromatic hydrocarbon groups such as phenyl groups, and aralkyl groups such as benzyl groups.

Examples of the thiuram disulfide-based vulcanization accelerator include tetramethylthiuram disulfide, tetraethylthiuram ethyldisulfide, tetrabutylthiuram disulfide, and tetrabenzylthiuram disulfide.

Among these, the thiuram disulfide-based vulcanization accelerator preferably includes an aralkyl group as R⁵ to R⁸, and more preferably a benzyl group (e.g. TbZTD manufactured by Flexsys Corp. as a commercially available product), from the perspective of exhibiting superior effect of the present invention.

The content of the thiuram disulfide-based vulcanization accelerator is preferably from 0.0 to 2.5 parts by mass and more preferably from 0.0 to 2.0 parts by mass per 100 parts by mass of the rubber component.

In one of the preferred aspects of the rubber composition of the present invention, the rubber composition may not substantially include a thiuram disulfide-based vulcanization accelerator from the perspective of superior processability. The expression “may not substantially include a thiuram disulfide-based vulcanization accelerator” means that the content of the thiuram disulfide-based vulcanization accelerator is from 0 to 0.1 parts by mass per the total of the composition.

### Additives

The rubber composition of the present invention may further contain additives as necessary within a scope that does not inhibit the effect or purpose thereof. Examples of the additive include various additives typically used in rubber compositions for a tire such as rubber other than diene rubber, silane coupling agents other than the polysiloxane above, fillers other than silica (e.g. carbon black, clay, mica, talc, calcium carbonate, aluminum hydroxide, aluminum oxide, and titanium oxide), vulcanization accelerator other than thiuram disulfide-based vulcanization accelerator, resin other than terpene resin, zinc oxide, stearic acid, anti-aging agents, processing aids, oils (e.g. aroma oils, process oils), liquid polymers, thermosetting resins, and vulcanizing agents such as sulfur. The content of the additives may be selected as desired.

The method of producing the rubber composition of the present invention is not particularly limited, and specific examples thereof include a method whereby each of the above-mentioned components is kneaded using a publicly known method and device (e.g. Banbury mixer, kneader, and roller).

In addition, the rubber composition of the present invention can be vulcanized or crosslinked under conventionally known vulcanizing or crosslinking conditions.

A tire can be produced by using the rubber composition of the present invention.

### Tire

The tire of the present invention is a tire in which the rubber composition of the present invention is used.

The rubber composition used in production of the tire of the present invention is not particularly limited as long as it is the rubber composition of the present invention.

The portion to which the rubber composition is applied is not particularly limited. Examples of the portions of the tire produced using the rubber composition include a tire tread, a bead portion, and a sidewall portion.

One of the preferable aspects of the tire of the present invention is a pneumatic tire.

The tire of the present invention will be described hereinafter with reference to the attached drawings. The tire of the present invention is not limited to the attached drawings.

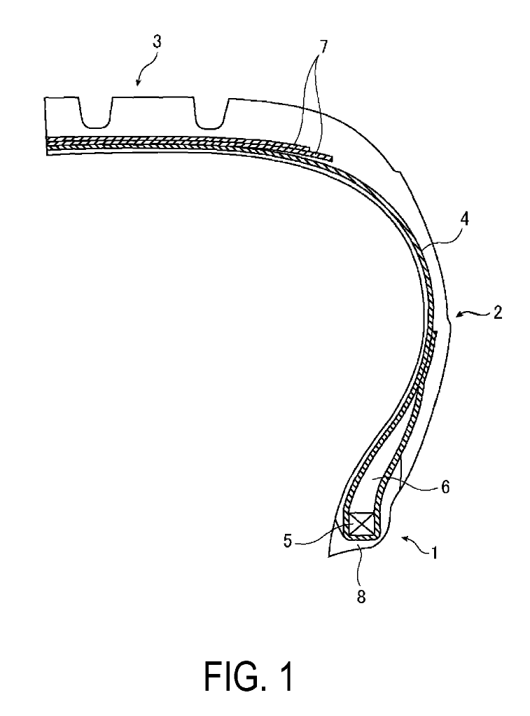

FIG. 1 is a partial cross-sectional schematic view of a tire that represents one embodiment of the tire of the present invention. Specifically, the tire illustrated in FIG. 1 is a pneumatic tire.

In FIG. 1, reference sign 1 denotes a bead portion, reference sign 2 denotes a sidewall portion, and reference sign 3 denotes a tire tread portion.

In addition, a carcass layer 4, in which a fiber cord is embedded, is mounted between a left-right pair of bead portions 1, and ends of the carcass layer 4 are wound by being folded around bead cores 5 and a bead filler 6 from an inner side to an outer side of the tire.

In the tire tread portion 3, a belt layer 7 is provided along the entire circumference of the tire on the outer side of the carcass layer 4.

Additionally, rim cushions 8 are provided in parts of the bead portions 1 that are in contact with a rim.

The tire of the present invention can be produced, for example, in accordance with a conventionally known method.

When the tire of the present invention is a pneumatic tire, in addition to ordinary air or air with an adjusted oxygen partial pressure, inert gases such as nitrogen, argon, and helium can be used as the gas with which the pneumatic tire is filled.

### Examples

The present invention is described below in detail using examples but the present invention is not limited to such examples.

### Production Method of Polysiloxane 1

107.8 g (0.2 mol) of bis(triethoxysilylpropyl)tetrasulfide (KBE-846, manufactured by Shin-Etsu Chemical Co., Ltd.), 190.8 g (0.8 mol) of γ-mercaptopropyl triethoxysilane (KBE-803, manufactured by Shin-Etsu Chemical Co., Ltd.), 442.4 g (1.6 mol) of octyl triethoxysilane (KBE-3083, manufactured by Shin-Etsu Chemical Co., Ltd.), and 190.0 g of ethanol were placed in a 2 L separable flask provided with an agitator, a reflux condenser, a dropping funnel and a thermometer, and then a mixed solution containing 37.8 g (2.1 mol) of 0.5 N hydrochloric acid and 75.6 g of ethanol was added in a dropwise manner at room temperature. It was then stirred for 2 hours at 80° C. Then, it was filtered, and 17.0 g of 5% KOH/EtOH solution was added in a dropwise manner, and stirred for 2 hours at 80° C. Then, by vacuum concentration and filtration, 480.1 g of polysiloxane in the form of a brown transparent liquid was obtained. As a result of performing measurements by GCP, the weight average molecular weight of the obtained polysiloxane was 840, and the average degree of polymerization was 4.0 (preset degree of polymerization: 4.0). In addition, as a result of measuring the mercapto equivalent weight of the obtained polysiloxane by an acetic acid/potassium iodide/potassium iodate addition/sodium thiosulfate solution titration method, the mercapto equivalent weight was 730 g/mol, and it was thus confirmed that the preset mercapto group content was achieved. The polysiloxane obtained as described above is represented by the following average composition formula.

(—C₃H₆—S₄—C₃H₆-)_{0.071}(—C₈H₁₇)_{0.571}(—OC₂H₅)_{1.50}(—C₃H₆SH)_{0.286}SiO_{0.75}

The obtained polysiloxane was used as polysiloxane 1.

### Synthesis of Nitrone Compound 1

In a 2 L eggplant-shaped flask, methanol heated to 40° C. (900 mL) was placed, and then terephthalaldehydic acid represented by Formula (b-1) below (30.0 g) was added and dissolved. To this solution, a solution in which phenylhydroxylamine represented by Formula (a-1) below (21.8 g) was dissolved in methanol (100 mL) was added and stirred at room temperature for 19 hours. After the completion of stirring, a nitrone compound (carboxynitrone) represented by Formula (c-1) below was obtained by recrystallization from methanol (41.7 g). The yield was 86%. The obtained nitrone compound is referred to as nitrone compound 1. The molecular weight of the nitrone compound 1 was 241.

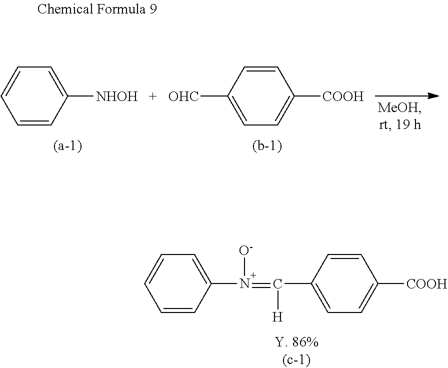

### Production of Modified Diene Rubber 1

137.5 parts by mass of the raw material SBR (styrene-butadiene rubber, trade name E581, oil extender content per 100 parts by mass of the net SBR: 37.5 parts by mass, weight average molecular weight: 1200000, styrene content: 37 mass %, vinyl bond content: 43%, manufactured by Asahi Kasei Corporation) and the nitrone compound 1 (1 part by mass) were mixed under the condition of 160° C. for 5 minutes in a mixer. Thus, the modified diene rubber 1, which was the raw material SBR modified by the nitrone compound 1, was obtained.

In the production above, 0.22 mol % of the total double bonds present in the raw material SBR was modified to carboxy groups by the nitrone compound 1.

The modified diene rubber 1 had a double bond and a carboxy group, and the content of the carboxy group was 0.22 mol % of the total of the double bonds and carboxy groups.

The degree of modification of the modified diene rubber 1 was 0.22 mol %.

### Production of Modified Diene Rubber 2

The modified diene rubber 2 was obtained in the same manner as the modified diene rubber 1 except that the used amount of the nitrone compound 1 was changed to 2 parts by mass.

In the production above, 0.43 mol % of the total double bonds present in the raw material SBR was modified to a carboxy group by the nitrone compound 1.

The modified diene rubber 2 had a double bond and a carboxy group, and the content of the carboxy group was 0.43 mol % of the total of the double bonds and carboxy groups.

The degree of modification of the modified diene rubber 2 was 0.43 mol %.

Production of the Rubber Composition

The components shown in Table 1 below were used in the amounts shown in the table (units: parts by mass), and these components were blended to produce a rubber composition. Specifically, the components shown in Table 1 below except sulfur and the vulcanization accelerator (DPG, CZ, TbZTD) were first mixed in a Banbury mixer at a temperature of 80° C. for 5 minutes, and the mixture was obtained. Thereafter, a roll was used to add the sulfur and the vulcanization accelerator to the mixture to obtain a rubber composition.

Note that when the amount of each modified diene rubber used in the Table 1 is 48.15 parts by mass, the net content of each modified diene rubber is 35 parts by mass. Alternatively, when the amount of the modified diene rubber used is 96.3 parts by mass, the net content of the modified diene rubber 1 is 70 parts by mass.

The content (CPN amount) of the nitrone compound 1 included in 35 parts by mass (net) of the modified diene rubber 1 is 0.32 parts by mass.

The content (CPN amount) of the nitrone compound 1 included in 70 parts by mass (net) of the modified diene rubber 1 is 0.64 parts by mass.

The content (CPN amount) of the nitrone compound 1 included in 35 parts by mass (net) of the modified diene rubber 2 is 0.64 parts by mass.

### Preparation of Vulcanized Rubber Sheet

A vulcanized rubber sheet was prepared by press-vulcanizing the prepared (unvulcanized) rubber composition prepared as above for 20 minutes at 160° C. in a mold (15 cm×15 cm×0.2 cm).

### Evaluation

The following evaluations were performed using the rubber composition and the vulcanized rubber sheet produced as described above. The results are shown in Table 1. Each result was shown as an index value, with the result of Comparative Example 1 expressed as 100.

Wet performance: tan δ (0° C.)

For the vulcanized rubber sheet produced as described above, loss tangent tan δ (0° C.) was measured using a viscoelastic spectrometer (manufactured by Toyo Seiki Seisaku-sho, Ltd.) in accordance with JIS K6394:2007 under the following conditions: a strain of tensile deformation of 10%±2%; an amplitude of ±2%; a frequency of 20 Hz; and a temperature of 0° C.

Larger indexes indicate larger tan δ (0° C.) values, which in turn indicate excellent wet performance.

### Wear Resistance

Wear resistance of the obtained vulcanized rubber produced as described above was measured in accordance with JIS K6264, using a Lambourn abrasion test machine (manufactured by Iwamoto Seisakusho Co. Ltd.) under the following conditions: a temperature of 20° C.; a load of 15 N; a slip rate of 50%; a duration of 10 minutes.

Note that the evaluation result of wear resistance was a reciprocal of the amount of wear of each sample, and shown as an index value, with the reciprocal of the amount of wear of Comparative Example 1 expressed as 100. Larger index values indicate smaller amount of wear, and thus excellent wear resistance of a formed tire.

### Mooney Scorch (Indicator of Scorch Resistance)

The Mooney scorch time (t₅) of the rubber composition (unvulcanized) produced as described above was measured under the conditions of a test temperature of 125° C. using an L-shaped rotor in accordance with JIS K6300-1:2001.

Larger Mooney scorch indexes indicate longer Mooney scorch times, which in turn indicates excellent scorch resistance (processability).

### t₉₅ (Index of Vulcanization Rate During Vulcanization)

The time t₉₅ (minutes) of the rubber composition for a tire produced as described above was measured using the vibration-type disk vulcanization tester, under the conditions of an amplitude of 1 degree and a temperature of 160° C., in accordance with JIS K6300.

A smaller value of t₉₅ (T95 in the Table) index indicates higher vulcanization rate and excellent vulcanization characteristics.

TABLE 1

| | Comparative | Example | Example | Example | Comparative | Example |
|---|---|---|---|---|---|---|
| | Example 1 | 1 | 2 | 3 | Example 2 | 4 |
| Solution-polymerized | 96.30 | 48.15 | | 48.15 | 96.30 | 48.15 |
| SBR (St 37%, Vi 43%, | | | | | | |
| Tg −27° C., 37.5 phr | | | | | | |
| oil extension) | | | | | | |
| BR | 30.00 | 30.00 | 30.00 | 30.00 | 30.00 | 30.00 |
| Modified diene rubber 1 | | 48.15 | 96.30 | | | 48.15 |
| Modified diene rubber 2 | | | | 48.15 | | |
| Silica | 110.00 | 110.00 | 110.00 | 110.00 | 110.00 | 110.00 |
| CB | 10.00 | 10.00 | 10.00 | 10.00 | 10.00 | 10.00 |
| Polysiloxane 1 | 8.80 | 8.80 | 8.80 | 8.80 | 8.80 | 8.80 |
| Stearic acid | 2.50 | 2.00 | 2.00 | 2.00 | 2.50 | 2.00 |
| Anti-aging agent | 2.00 | 2.00 | 2.00 | 2.00 | 2.00 | 2.00 |
| Oil | 10.00 | 10.00 | 10.00 | 10.00 | 10.00 | 10.00 |
| Zinc white | 2.50 | 2.50 | 2.50 | 2.50 | 2.50 | 2.50 |
| Sulfur | 2.00 | 2.00 | 2.00 | 2.00 | 2.00 | 2.00 |
| DPG | | | | | | |
| CZ | 2.00 | 2.00 | 2.00 | 2.00 | 2.00 | 2.00 |
| TbZTD | | | | | 0.30 | 0.30 |
| CPN amount | 0.00 | 0.32 | 0.64 | 0.64 | 0.00 | 0.32 |
| Wet performance | 100 | 103 | 102 | 103 | 102 | 105 |
| Wear resistance | 100 | 113 | 123 | 122 | 103 | 116 |
| Mooney scorch | 100 | 104 | 106 | 104 | 91 | 100 |
| T95 | 100 | 97 | 88 | 82 | 88 | 85 |

Details of the components described in Table 1 are as follows.

TABLE 2

| | Details of each component | |
|---|---|---|
| Solution-polymerized SBR | Manufactured by Asahi | E581: Styrene-butadiene rubber, oil |
| (St 37%, Vi 43%, Tg −27° C., | Kasei Chemicals | extending quantity per 100 parts by mass |
| 37.5 phr oil | Corporation | of the net SBR component: 37.5 parts by |
| extension) | | mass (net SBR content per 96.3 parts by |
| | | mass: 70 parts by mass), weight average |
| | | molecular weight: 1200000, styrene |
| | | content: 37 mass %, vinyl bond content: |
| | | 43%, glass transition temperature: −27° C. |
| BR | Manufactured by Zeon | Trade name A1220, Butadiene rubber |
| | Corporation | |
| Modified diene rubber 1 | Modified diene rubber 1 produced as described above | |
| Modified diene rubber 2 | Modified diene rubber 2 produced as described above | |
| Silica | Manufactured by | Zeosil 1165MP; the silica having CTAB |
| | Rhodia | specific surface area of 159 m²/g |
| CB | Manufactured by Cabot | Show Black N339, Carbon black |
| | Japan K. K. | |
| Polysiloxane 1 | Polysiloxane 1 produced as described above | |
| Stearic acid | Manufactured by NOF | Stearic acid YR |
| | Corporation | |
| Anti-aging agent | Manufactured by | Santoflex 6PPD (N-phenyl-N′-(1,3- |
| | Flexsys | dimethylbutyl)-p-phenylenediamine) |
| Oil | Showa Shell Sekiyu | Extract No. 4S |
| | KK | |
| Zinc oxide | Seido Chemical | Zinc Oxide III |
| | Industry Co., Ltd. | |
| Sulfur | Karuizawa Seirensho | Oil treatment sulfur |

| | |
|---|---|
| DPG | Vulcanization accelerator: 1,3-diphenylguanidine (Soxinol D-G, |
| | manufactured by Sumitomo Chemical Co., Ltd.) |
| CZ | Vulcanization accelerator: N-cyclohexyl-2-benzothiazolyl |
| | sulfenamide (NOCCELER CZ-G, manufactured by Ouchi Shinko |
| | Chemical Industrial Co., Ltd.) |
| TbZTD | Vulcanization accelerator: tetrabenzylthiuram disulfide, |
| | manufactured by Flexsys |

As shown in Table 1, Comparative Example 2 which did not include the modified diene rubber exhibited lower processability and more room for wear resistance improvement compared to Comparative Example 1.

In contrast, the desired effects were confirmed for the rubber composition of the present invention. Specifically, Examples 1 to 4 exhibited better wear resistance than Comparative Example 2 while maintaining an excellent wet performance. They also exhibited superior processability.

In particular, when Examples 1 to 3 were compared with regard to CPN amount, Examples 2 and 3, which included a larger CPN amount than Example 1, were confirmed to exhibit superior wear resistance than Example 1.

When Examples 1 to 3 were compared with regard to the degree of modification of the modified diene rubber, Examples 3, which showed a degree of modification higher than Examples 1 and 2, was confirmed to exhibit smaller t₉₅ and superior vulcanization rate than Example 1.

With regard to the presence or absence of the thiuram disulfide-based vulcanization accelerator, comparison between Example 1 and 4 confirmed Example 1, which did not contain the thiuram disulfide-based vulcanization accelerator, exhibited better processability than Example 4.

Furthermore, it was confirmed that Example 4, which included thiuram disulfide-based vulcanization accelerator, showed better wet performance and wear resistance and superior vulcanization rate than Example 1.

### REFERENCE SIGNS LIST

1 Bead portion
2 Sidewall portion
3 Tire tread portion
4 Carcass layer
5 Bead core
6 Bead filler
7 Belt layer
8 Rim cushion

1. A rubber composition for a tire comprising:
a rubber component comprising a modified diene rubber, the modified diene rubber having from 0.2 to 4 mol % of all the double bonds in a raw material diene rubber modified by a carboxy group;
a silica; and
polysiloxane represented by an average compositional formula (I);
wherein a content of the silica is from 60 to 200 parts by mass per 100 parts by mass of the rubber component, a content of the polysiloxane is from 1 to 20 mass % based on the content of the silica, and a content of the modified diene rubber is from 10 to 100 parts by mass per 100 parts by mass of the rubber component;
(A)ₐ(B)_{b}(C)_{c}(D)_{d}(R¹)ₑSiO_{(4-2a-b-c-d-e)/2} (I)
where, in the average compositional formula (I), A is a divalent organic group containing a sulfide group; B is a monovalent hydrocarbon group having from 5 to 10 carbons; C is a hydrolyzable group; D is an organic group containing a mercapto group; R¹ is a monovalent hydrocarbon group having from 1 to 4 carbons; and a to e satisfy the relational expressions 0≤a<1, 0≤b<1, 0<c<3, 0<d<1, 0≤e<2, and 0<2a+b+c+d+e<4, and at least one of a and b is not 0.

2. The rubber composition according to claim 1, wherein the modified diene rubber is produced by a reaction between the raw material diene rubber and a nitrone compound having a carboxy group and a nitrone group.

3. The rubber composition according to claim 2, wherein
the nitrone compound is at least one type selected from the group consisting of N-phenyl-α-(4-carboxyphenyl)nitrone, N-phenyl-α-(3-carboxyphenyl)nitrone, N-phenyl-α-(2-carboxyphenyl)nitrone, N-(4-carboxyphenyl)-α-phenylnitrone, N-(3-carboxyphenyl)-α-phenylnitrone, and N-(2-carboxyphenyl)-α-phenylnitrone.

4. The rubber composition according to claim 2, wherein a content of the nitrone compound introduced in the modified diene rubber is not less than 0.3 parts by mass and not greater than 10 parts by mass per 100 parts by mass of the rubber component.

5. A rubber composition for a tire comprising:
a rubber component comprising a modified diene rubber, the modified diene rubber having a double bond and a carboxy group, and a content of the carboxy group being from 0.2 to 4 mol % of a total of the double bond and the carboxy group;
a silica; and
polysiloxane represented by an average compositional formula (I);
wherein a content of the silica is from 60 to 200 parts by mass per 100 parts by mass of the rubber component, a content of the polysiloxane is from 1 to 20 mass % based on the content of the silica, and a content of the modified diene rubber is from 10 to 100 parts by mass per 100 parts by mass of the rubber component;
(A)ₐ(B)_{b}(C)_{c}(D)_{d}(R¹)ₑSiO_{(4-2a-b-c-d-e)/2} (I)
where, in the average compositional formula (I), A is a divalent organic group containing a sulfide group; B is a monovalent hydrocarbon group having from 5 to 10 carbons; C is a hydrolyzable group; D is an organic group containing a mercapto group; R¹ is a monovalent hydrocarbon group having from 1 to 4 carbons; and a to e satisfy the relational expressions 0≤a<1, 0≤b<1, 0<c<3, 0<d<1, 0≤e<2, and 0<2a+b+c+d+e<4, and at least one of a and b is not 0.

6. A tire comprising the rubber composition described in claim 1.
